(19) United States
(12) Patent Application Publication
(10) Pub. No.: US 2019/0358043 A1
(43) Pub. Date: Nov. 28, 2019

(54) Artificial hip joint comprising composite ball

(71) Applicants: Guoan Li, Milton, MA (US); Jianguo Li, Nanchang, Jiangxi (CN)

(72) Inventors: Guoan Li, Milton, MA (US); Jianguo Li, Nanchang, Jiangxi (CN)

(21) Appl. No.: 16/477,118

(22) PCT Filed: Jan. 3, 2018
(86) PCT No.: PCT/CN2018/070086

§ 371 Date: Aug. 12, 2019

(30) Foreign Application Priority Data
Jan. 10, 2017 (CN) 201710016927.8

(51) Int. Cl. A61F 2/36; A61F 2/34; A61L 27/04; A61L 27/34

(52) U.S. Cl. CPC A61F 2/3609; A61F 2/34; A61F 2002/3065; A61L 27/34; A61F 2002/3483; A61L 27/04

(57) ABSTRACT

An artificial hip joint with a composite ball head includes an artificial acetabulum (1) and a composite ball head (2); wherein an internal surface of the artificial acetabulum (1) cooperates with an external surface of the composite ball head (2), and the composite ball head (2) can rotate within the artificial acetabulum (1); the internal surface of the artificial acetabulum (1) is directly in contact with the composite ball head (2) without a liner therebetween; the composite ball head (2) comprises a metal ball head (22) and a non-metallic shell (21) wrapped around an external surface of the metal ball head (22). For the composite ball head (2), the metal ball head (22) is wrapped with a non-metallic shell (21), which has large supporting force capability, has strong bearing capacity, effectively reduces the contact stress of the non-metallic shell (21), is stable and reliable.

### CROSS REFERENCE OF RELATED APPLICATION

This is a U.S. National Stage under 35 U.S.C. 371 of the International Application PCT/CN2018/070086, filed Sep. 19, 2018, which claims priority under 35 U.S.C. 119(a-d) to CN 201710016927.8, filed Jan. 10, 2017.

### BACKGROUND OF THE PRESENT INVENTION

### Field of Invention

The present invention relates to a field of medical instruments, and more particularly to an artificial hip joint with a composite ball head.

### Description of Related Arts

The human hip joint consists of acetabulum and femoral ball head. The ball head can rotate in the acetabulum to achieve normal movement of the hip joint. The human femoral ball head has a diameter of about 40 to 45 mm. When the hip joint is damaged, such as hip osteoarthritis, cartilage lesions or femoral neck rupture that causes the hip joint not functioning properly, hip joint replacement is required, that is, the artificial hip joint is placed through orthopedic surgery (see FIG. 1), which is the most commonly used surgical treatment method.

The conventional artificial hip joint is usually composed of a metal ball head, a metal artificial acetabulum and a non-metallic (usually high molecular weight polyethylene material) acetabular shell liner (see FIG. 2). Due to the limited strength of the polyethylene material, there is a certain requirement for the thickness of the liner. Thus, the thickness of the acetabular shell plus the thickness of the liner makes the inner diameter of the liner smaller than the inner diameter of human acetabulum. Accordingly, the diameter of the metal ball head is smaller than the diameter of the human femoral ball head. As a result, the contact area between the ball head and the liner is reduced, the contact stress is increased, and the wear of the liner is increased in practical use. At the same time, due to the reduction of the diameter of the ball head, the range of motion of the hip joint is reduced, affecting the normal life of the patient. Furthermore, it is easy to cause the femoral neck to collide with the liner boundary and cause the metal ball head to contact the liner boundary, resulting in problems such as high boundary stresses (edge loading) and damage to the liner material, and leading to failure of the artificial joint and thus revision surgery.

In order to solve these problems, ceramic materials have been introduced into artificial hip joints due to their high strength and wear resistance. However, due to the brittleness of the ceramic material, there is a certain requirement on the thickness of the liner, and the size of the ball head is still limited. The negative effects of the small ball head still exist, and the ceramics are prone to sharp noise due to friction, which is inconvenient for the patient's life and still needs to be greatly improved.

If the liner is not used and the metal ball head is used in combination with the metal shell, the metal ball head size can be made larger, making the large ball head possible. However, metal scraps and metal ions generated by metal friction are harmful to human body, so the design of metal ball head with metal shell has been abandoned by the clinic.

Chinese patent CN201610325901.7 discloses a non-metallic hip joint prosthesis comprising an acetabular shell prosthesis, a non-metallic femoral head prosthesis and a femoral stem prosthesis, which reduces the thickness of the acetabular shell and allows the same-diameter acetabular shell to pair with a large-diameter ball head. However, the non-metallic femoral head prosthesis is difficult to achieve in practice, because sharp corners exist between the non-metallic femoral head prosthesis and the metal femoral stem that can lead to stress concentration in the non-metallic femoral head prosthesis and reduce its structural strength. It is difficult to carry the weight of the human body, and can be easily damaged once it is used in the artificial hip joint.

Therefore, development of a clinically reliable large ball head artificial hip joint is still the objective of orthopedic bioengineering.

### SUMMARY OF THE PRESENT INVENTION

To overcome the deficiencies of the prior art, an objective of the present invention is to provide an artificial hip joint with a composite ball head, wherein no liner is used in the acetabulum so that the accommodating space of the acetabular shell is increased and the size of the composite ball head can be increased to increase the joint mobility and truly realize the advantages of the large ball head hip joint; at the same time, for the composite ball head, the metal ball head is wrapped with a non-metallic shell, which has large supporting area, reduces the contact stress of the material, has strong bearing capacity, is not easy to be damaged, effectively extends the service life of artificial joints, and enhances the usage of artificial joints.

Accordingly, in order to accomplish the above object, the present invention provides an artificial hip joint, comprising of an artificial acetabulum and a composite ball head; wherein an internal surface of the artificial acetabulum interacts with an external surface of the composite ball head, and the composite ball head can rotate within the artificial acetabulum; the internal surface of the artificial acetabulum is directly in contact with the composite ball head without a liner therebetween; the composite ball head is comprised of a metal ball head and a non-metallic shell wrapped around an external surface of the metal ball head.

Preferably, the metal ball head has a concave tapered space which engages with a top end of a femoral stem, and the metal ball head is combined with the femoral stem through the concave tapered space, so as to be installed at a thigh bone.

Alternatively, the metal ball head of the composite ball head is made one piece with a top end of a femoral stem. As a result, the composite ball head is integrated with the femoral stem through the metal ball head as one piece, including the composite ball head, the top end of the femoral stem and the femoral stem.

Preferably, the artificial acetabulum is made of a metal material.

Preferably, the non-metallic shell of the composite ball head is made of a wear resistant non-metallic material.

Preferably, the non-metallic shell of the composite ball head is made of a high molecular weight polyethylene material or a PEEK (polyether ether ketone) material.

Preferably, the internal surface of the artificial acetabulum is a smooth surface. The internal surface refers to a concave spherical surface of the artificial acetabulum.

Preferably, an external surface of the artificial acetabulum is coated with a coating which facilitates bone ingrowth. The external surface refers to a convex spherical surface of the artificial acetabulum.

Preferably, a size of the composite ball head is close to, identical to, or exceeds a size of a patient femoral head matching to a size of the artificial acetabulum.

Preferably, the metal ball head comprises a protruding member, and the protruding member is in contact with an external bottom end of the non-metallic shell.

Preferably, the metal ball head comprises a spherical end portion and a cylindrical extension portion; a cavity formed by the internal surface of the non-metallic shell matches with an external shape of the metal ball head.

The present invention adopts the metal material to make the artificial acetabulum, and the internal surface of the acetabulum is smooth, so that the composite ball head can freely rotate therein. Since no liner is used, the metal acetabulum is thin and the acetabular shell has a larger accommodation space. Since the thickness of the acetabulum can be made thinner, the acetabular shell has a larger accommodation space to accommodate the composite ball head. Therefore, the size of the composite ball head can be close to or exceed the actual size of the patient's femoral ball head according to the size of the acetabular shell, so as to achieve a true large ball head artificial hip joint.

Furthermore, the external surface of the metal artificial acetabulum may be coated with the coating that facilitates bone ingrowth.

The composite ball head of the present invention is formed by wrapping the external surface of the metal ball head with the non-metallic (high molecular weight polyethylene or PEEK or other wear-resistant non-metal) shell. The external surface of the metal ball head completely matches with the internal surface of the non-metallic shell without relative movement. The external surface of the non-metallic shell forms a pair of sliding surfaces with the smooth internal surface of the metal artificial acetabulum, and the composite ball head can freely rotate on the internal surface of the artificial acetabulum. The non-metallic shell made of wear-resistant non-metallic material avoids the brittleness of the ceramic material while has high strength, high toughness and excellent wear resistance. The metal ball is within the non-metallic shell, in such a manner that the composite ball head has an increased strength, a strong bearing capacity, and is not easy to be damaged.

One implementation manner of the present invention is that the metal ball head of the composite ball head has the concave tapered space matching the top end of the femoral stem, and the metal ball head can be combined with the femoral stem through the concave tapered space and installed at the thigh bone. Such a connection method is stable and reliable in practical applications and is not easy to damage. The femoral stem can be any commercially available femoral stem, and the concave tapered space is formed to fit the top of the femoral stem. Therefore, the present invention can be combined with any conventional femoral stem and is easy to implement, providing a wide range of versatility.

Another implementation of the present invention is that the metal ball head can also be designed and manufactured integrally with the femoral stem, and is provided as a complete elongated metal femoral stem (including the metal ball head, the top end of the femoral stem and the femoral stem), which completely eliminates the contact between the metal ball head and the metal femoral stem. As a result, the artificial hip joint of the present invention does not introduce any metal-to-metal interface, and thereby completely eliminates the origin of metal debris and metal ions.

The artificial hip joint with the composite ball head of the present invention does not use a liner in the acetabulum, and the accommodation space of the acetabular shell is increased, wherein the liner of the acetabular shell is made into the external shell of the metal ball head, and is directly combined with the metal ball head to form the composite ball head which is used in combination with the metal acetabulum shell. In this way, the size of the composite ball head can be increased to close to or exceed the size of the patient's femoral ball head, thereby increasing the joint mobility, reducing edge loading, reducing the collision between boundaries of the femoral stem and the acetabular shell, reducing the risk of dislocation of the acetabulum and the ball head, avoiding the negative effects of the small ball head, effectively prolonging the service life of the artificial joint, and enhancing the functional usage of the artificial joint. Compared with the conventional surgery, it is easy to implant without increasing any difficulty.

The present invention adopts the composite ball head, and the metal ball head is wrapped with the wear-resistant non-metal shell, wherein the support area is large, the contact stress is reduced, the bearing capacity is strong, stable and reliable. It is not easy to be damaged, and the service life of the artificial joint is prolonged.

The present invention can be used in conjunction with any conventional femoral stem, which is easy to implant, and has a wide range of versatility. The present invention can also completely eliminate the contact surface between the metal ball head and the metal femoral stem, and does not introduce any metal-to-metal interface, thereby completely eliminating the origin of metal debris and metal ions.

### BRIEF DESCRIPTION OF THE DRAWINGS

FIG. 1 is a sketch view of artificial hip joint replacement; wherein 1—artificial acetabulum, 2—artificial ball head, 3—femoral stem, 4—femur, 5—pelvis;

FIG. 2 is a sketch view of a conventional artificial hip joint; wherein 1—artificial acetabulum, 1—artificial acetabular shell liner, 2—artificial ball head, 3—femoral stem;

FIG. 3 is a sketch view of an artificial hip joint of the present invention; wherein 1—artificial acetabulum, 2—composite ball head, 21—non-metallic shell, 22—metal ball head, 3—femoral stem;

FIG. 4 is a structural view according to an embodiment 1 of the present invention; wherein 1—artificial acetabulum, 21—non-metallic shell, 22—metal ball head, 23—concave tapered space, 3—femoral stem;

FIG. 5 is a structural view according to an embodiment 2 of the present invention; wherein 1—artificial acetabulum, 21—non-metallic shell, 22—metal ball head, 3—femoral stem;

FIG. 6 is a structural view according to an embodiment 3 of the present invention; wherein 1—artificial acetabulum, 21—non-metallic shell, 22—metal ball head, 24—protruding member;

FIG. 7 is an exploded view according to an embodiment 3 of the present invention; wherein 1—artificial acetabulum, 21—non-metallic shell, 22—metal ball head, 24—protruding member, 3—femoral stem;

FIG. 8 illustrates comparison of active angles of the present invention and a conventional small spherical head hip joint; wherein a illustrates the active angle of the conventional small spherical head hip joint; wherein a-1: artificial acetabulum, a-11: artificial acetabular shell liner, a-2: artificial ball head, a-3: femoral stem; b illustrates the active angle of the artificial hip joint of the present invention; wherein b-1: artificial acetabulum, b-2: composite ball head, b-3: femoral stem;

A: increment of active angle of the present invention;

FIG. 9 illustrates comparison of maximum stress of the present invention and a conventional metal ball joint artificial hip joint, wherein a illustrates a conventional metal ball head hip joint, and b illustrates the artificial hip joint of the present invention.

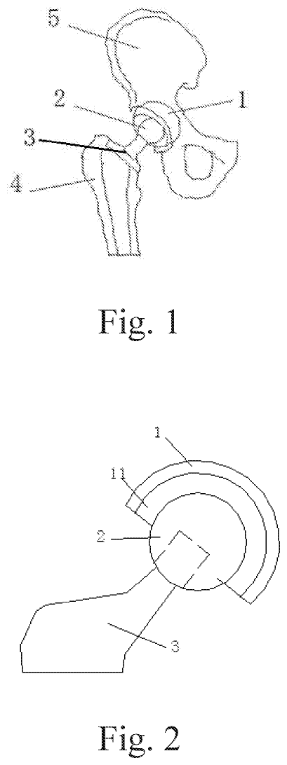

### DETAILED DESCRIPTION OF THE PREFERRED EMBODIMENT

The technical solutions in the embodiments of the present invention will be clearly and completely described below. It is apparent that the described embodiments are only a part of the embodiments of the present invention, and not all of the embodiments. All other embodiments obtained by those skilled in the art based on the embodiments of the present invention without creative efforts are within the scope of the present invention.

A conventional artificial hip joint usually comprises a metal artificial acetabulum 1 and an artificial ball head 2, wherein an internal surface of the artificial acetabulum 1 is provided with a non-metal (usually made of high molecular weight polyethylene material) acetabular shell liner 11, as shown in FIG. 2. Due to the limitations of the design, the acetabular shell liner 11 usually needs a certain thickness, so that the cavity formed on the internal surface of the acetabular shell liner 11 has a small volume, which limits the diameter of the metal ball head 2. Because the acetabular shell liner 11 and the artificial acetabulum 1 are relatively fixed to form an acetabular shell, and the metal ball head 2 is rotatable as a movable member in the acetabular shell, the smaller internal surface of the acetabular shell and the small-sized metal ball head will bring the aforementioned all kinds of drawbacks.

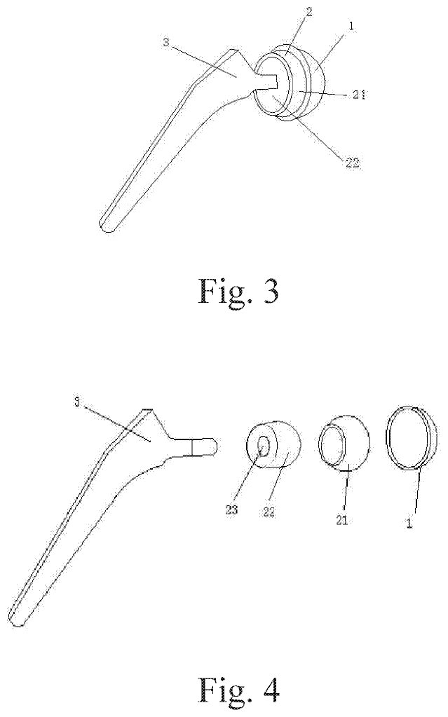

Therefore, the present invention provides an artificial hip joint with a composite ball head as shown in FIG. 3, comprising an artificial acetabulum 1 and the composite ball head 2; wherein an internal surface of the artificial acetabulum 1 cooperates with an external surface of the composite ball head 2, and the composite ball head 2 can rotate within the artificial acetabulum 1; the internal surface of the artificial acetabulum 1 is directly in contact with the composite ball head 2 without a liner therebetween; the composite ball head 2 comprises a metal ball head 22 and a non-metallic shell 21 wrapped around an external surface of the metal ball head 22.

Since no liner is used in the present invention, the metal acetabulum 1 is thinner and the acetabular shell formed at the internal surface thereof has a larger accommodation space. Since the thickness of the artificial acetabulum 1 can be made thinner, the acetabular shell has a larger accommodation space to accommodate the composite ball head 2. Therefore, the size of the composite ball head 2 can be close to or exceed the actual size of the patient's femoral ball head according to the size of the acetabular shell, so as to achieve a true large ball head artificial hip joint.

Referring to FIG. 3, the femoral stem 3 can be separately produced or purchased as a prior art, which is then assembled with the metal ball head 22 of the present invention. Referring to FIG. 4, the metal ball head 21 has a concave tapered space 23 which cooperates with a top end of a femoral stem 3, and the metal ball head 21 is combined with the femoral stem 3 through the concave tapered space 23, so as to be installed at a thigh bone.

Alternatively, the metal ball head of the composite ball head is integrally made with a top end of a femoral stem. As a result, the composite ball head is integrated with the femoral stem through the metal ball head as a single piece, which reduces the interface of the metal parts, thereby greatly reducing the risk of wear and metal powder due to contact between the metal parts.

In practical applications, the artificial acetabulum 1 is preferably made of a metallic material. Some metal materials have high strength, toughness and wear resistance, which can make the artificial acetabulum 1 thin enough to obtain a larger acetabular shell, thus bringing the possibility of using a large-sized composite ball head 2. In practical applications, the non-metallic shell 21 of the composite ball head 2 is made of a wear resistant non-metallic material.

In practical applications, the non-metallic shell of the composite ball head is made of a high molecular weight polyethylene material or a PEEK (polyether ether ketone) material.

In practical applications, the internal surface of the artificial acetabulum (namely the acetabular shell surface) is a smooth surface.

Preferably, an external surface of the artificial acetabulum 1 is coated with a coating which facilitates bone ingrowth. The external surface refers to a convex spherical surface of the artificial acetabulum.

Preferably, a size of the composite ball head is close to, identical to, or exceeds a size of a patient femoral head according to a size of the artificial acetabulum.

The present invention adopts the metal material to make the artificial acetabulum, and the internal surface of the acetabulum is smooth, so that the composite ball head can freely rotate therein. Since no liner is used, the metal acetabulum is thin and the acetabular shell has a large accommodation space. Since the thickness of the acetabulum can be made thinner, the acetabular shell has a larger accommodation space to accommodate the composite ball head. Therefore, the size of the composite ball head can be close to or exceed the actual size of the patient's femoral ball head according to the size of the acetabular shell, so as to achieve a true large ball head artificial hip joint.

The composite ball head of the present invention is formed by wrapping the external surface of the metal ball head with the non-metallic (high molecular weight polyethylene or PEEK or other wear-resistant non-metal) shell. The external surface of the metal ball head completely matches with the internal surface of the non-metallic shell without relative movement. The external surface of the non-metallic shell forms a pair of sliding surfaces with the smooth internal surface of the metal artificial acetabulum, and the composite ball head can freely rotate on the internal surface of the artificial acetabulum. The non-metallic shell made of wear-resistant non-metallic material avoids the brittleness of the ceramic material while has high strength, high toughness and excellent wear resistance. The metal ball is within the non-metallic shell, in such a manner that the composite ball head has an increased strength, a strong bearing capacity, and is not easy to be damaged.

One implementation manner of the present invention is that the metal ball head of the composite ball head has a concave tapered space matching the top end of the femoral stem, and the metal ball head can be combined with the femoral stem through the concave tapered space and install at the thigh bone. Such a connection method is stable and reliable in practical applications and is not easy to be damaged. The femoral stem can be any commercially available femoral stem, and the concave tapered space is formed to fit the top of the femoral stem. Therefore, the present invention can be combined with any conventional femoral stem and is easy to implement, providing a wide range of versatility.

Another implementation of the present invention is that the metal ball head can also be designed and manufactured integrally with the femoral stem, and is provided as a complete elongated metal femoral stem (including the metal ball head, the top end of the femoral stem and the femoral stem), which completely eliminates the contact between the metal ball head and the metal femoral stem. As a result, the artificial hip joint of the present invention does not introduce any metal-to-metal interface, and thereby completely eliminates the origin of metal debris and metal ions.

The artificial hip joint with the composite ball head of the present invention does not use liner in the acetabulum, and the accommodation space of the acetabular shell is increased, wherein the liner of the acetabular shell is made into the shell of the metal ball head, and is directly combined with the metal ball head to form the composite ball head which is used in combination with the metal shell. In this way, the size of the composite ball head can be increased to close to or exceed the size of the patient's femoral ball head, thereby increasing the joint mobility, reducing the boundary stress/edge loading, reducing the collision between boundaries of the femoral stem and the acetabular shell, reducing the risk of dislocation of the acetabulum and the ball head, avoiding the negative effects of the small ball head, effectively prolonging the service life of the artificial joint, and enhancing the functional usage of the artificial joint. Compared with the conventional surgery, it is easy to implant without increasing any difficulty.

The present invention adopts the composite ball head, and the metal ball head is wrapped with the wear-resistant non-metal shell, wherein the support area is large, the contact stress is reduced, the bearing capacity is strong, stable and reliable. It is not easy to be damaged, and the service life of the artificial joint is prolonged.

The present invention can be used in conjunction with any conventional femoral stem, which is easy to implement, and has a wide range of versatility. The present invention can also completely eliminate the contact surface between the metal ball head and the metal femoral stem, and does not introduce any metal-to-metal interface, thereby completely eliminating the origin of metal debris and metal ions.

Referring to FIG. 4, the composite ball head 2 comprises the non-metallic shell 21 and the metal ball head 22, wherein an end face of the metal ball head 22 that is not in contact with the non-metallic shell is provided with concave tapered space 23 that cooperates with a top end of the femoral stem 3. The top end of the femoral stem 3 is inserted into the concave tapered space 23 to join the femoral stem 3 to the composite ball head 2. The femoral stem 3 can be any femoral stem available on the market.

When in use, the metal artificial acetabulum can be mounted to the corresponding position of the pelvic acetabulum of the patient by squeezing/press fitting or cementing, and the composite ball head is attached to the thigh bone through the concave tapered space of the metal ball head and the femoral stem. Such a connection method is stable and reliable in practical applications and is not easy to be damaged.

The femoral stem can be any commercially available femoral stem, and the concave tapered space is formed to match the top of the femoral stem. Therefore, the present invention can be combined with any conventional femoral stem and is easy to implement, providing a wide range of versatility.

In some embodiments, the artificial hip joint with the composite ball head comprises an artificial acetabulum 1 and a composite ball head 2, wherein the artificial acetabulum 1 is made of a metal material, and the internal surface thereof is a smooth slip surface without a liner. The external surface of the artificial acetabulum 1 is coated to facilitate ingrowth of the bone after implantation. The composite ball head 2 comprises a metal ball head 22 made of a metal material and a non-metallic shell 21 wrapped around the metal ball head. The non-metallic shell is made of high molecular weight polyethylene or PEEK (polyether ether ketone) material, and the external surface of the metal ball head is tightly wrapped.

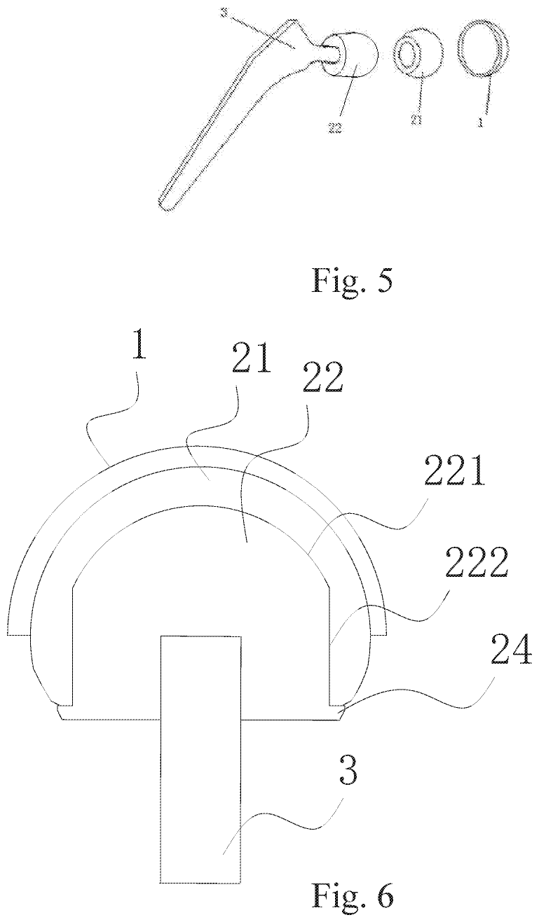

Referring to FIG. 5, the composite ball head 2 comprises the non-metallic shell 21 and the metal ball head 22. The metal ball head 22 is integrally formed with the top end of the femoral stem 3 to form a fully extended metal stem, thereby combining the femoral stem, the metal ball head, and the non-metallic shell. In use, the metal artificial acetabulum can be attached to the pelvic acetabulum of the patient by squeezing/press fitting or bone cement, and the composite ball head is attached to the thigh bone through a femoral stem integrated with the metal ball head.

In this embodiment, since the metal ball head and the top end of the femoral stem are integrally arranged, the metal-to-metal interface in the ball head is completely eliminated, and thereby completely eliminates metal debris and metal ions generated by the metal-to-metal interface of.

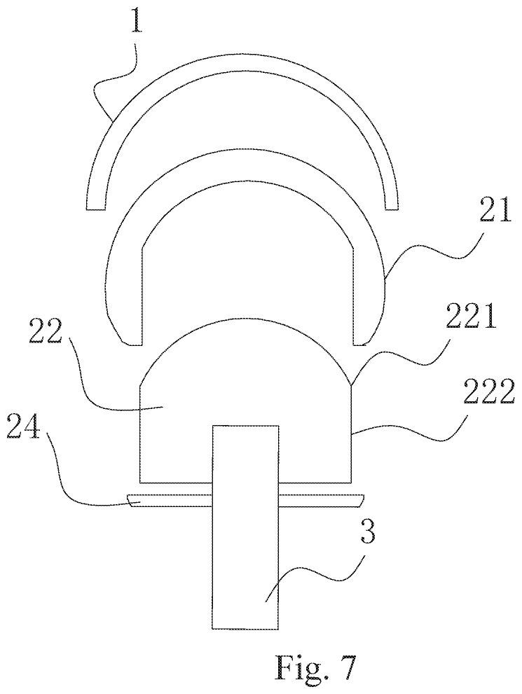

In some embodiments, a protruding member 24 that is in contact with the external bottom of the non-metallic shell 21 is provided in the bottom portion of the metal ball head 22, as shown in FIGS. 6 and 7. The protruding member 24 can enhance the stability of the non-metallic shell 21.

Referring to FIGS. 6 and 7, the metal ball head 22 comprises a spherical end portion 221 and a cylindrical protruding portion 222, wherein a cavity formed by the internal surface of the non-metallic shell 21 matches with an external shape of the metal ball head 22. With such structure, the connection tightness between the metal ball head 22 and the non-metallic shell 21 can be greatly improved, the slip between the two can be effectively prevented, and the stability of the product can be improved. A smooth curve may be used between the spherical end surface 221 and the cylindrical protruding portion 222. In some embodiments, the cross-sectional dimension of the cylindrical protruding portion 222 gradually decreases from the side near the spherical end surface 221 toward the side of the femoral stem 3, or gradually decreases in an undulating shape, which further improves the stability of the product.

Since the present invention has no liner, the acetabulum can be thin, the diameter of the composite ball head can be larger, the contact area between the ball head and the acetabulum is increased, the contact stress is reduced, and the active angle of the ball head is in the acetabular shell is large, which increases the range of motion of the hip joint.

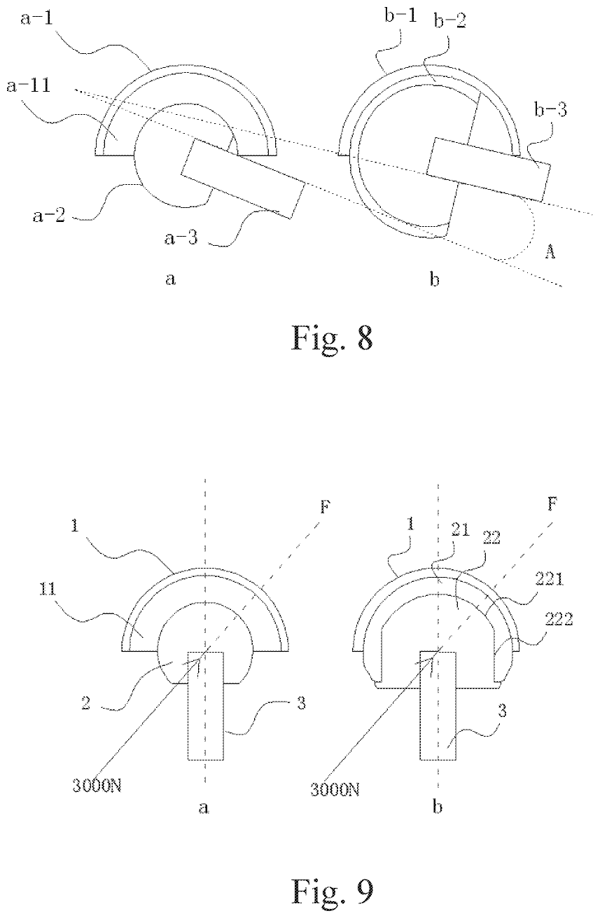

FIG. 8 illustrates comparison of active angles of the present invention and a conventional small spherical head hip joint; wherein a illustrates the active angle of the conventional small spherical head hip joint. The artificial acetabulum a-1 is provided with a liner a-11, the thickness of the acetabulum plus the thickness of the liner makes the inner diameter of the liner smaller than the inner diameter of human acetabulum. Correspondingly, the diameter of the metal ball head a-2 is reduced, causing the active angle of the metal ball head to decrease, which easily causes the collision between boundaries of the femoral stem a-3 and the liner a-11, as well as between boundaries of the metal ball head and the liner 11. Correspondingly, the range of motion of the hip joint is reduced. At the same time, since the diameter of the ball head is reduced, the contact area between the ball head and the liner is small, and the contact stress is increased. In actual use, liner wear is likely to be increased, and the service life of the joint is reduced. b of FIG. 8 illustrates the active angle of the artificial hip joint of the present invention; wherein the metal acetabulum b-1 is not provided with the liner, so the metal acetabulum is thinner and the acetabular shell has a larger accommodation space. Therefore, the composite ball head b-2 can be used and the size of the composite ball head can be close to or exceed the actual size of the patient's femoral ball head according to the size of the acetabular shell, thereby increasing the active angle and the range of motion of the hip joint. The angle A is increment of active angle of the present invention compared with the artificial hip joints. Due to the increased active angle and the range of motion of the hip joint according to the present invention, the present invention overcome the problems of small ball head hip joint in which the femoral neck collides with the liner boundary and the metal ball head contacts with the liner boundary, limiting the use of the hip joint and the realization of physiological functions. As a result, the service life of the artificial joint of the present invention will naturally increase. At the same time, the present invention adopts a composite ball head, and the metal ball head is wrapped with a wear-resistant non-metal shell, wherein the support area is large, the contact stress is reduced, the bearing capacity is strong, which is stable and reliable, and is not easy to be damaged.

FIG. 9 illustrates comparison of maximum stress of the present invention and a conventional metal ball joint artificial hip joint, wherein a illustrates a conventional metal ball head hip joint, and b illustrates the artificial hip joint of the present invention. F in FIG. 9 indicates the load direction at a certain angle. Table 1 shows comparison of the maximum stress of the present invention and the conventional metal ball head hip joint under the same load conditions.

TABLE 1
comparison of the maximum stress in the polyethylene component
of the present invention and the conventional metal ball head

| | Peak von Mises Stress (MPa) | | |
|---|---|---|---|
| Load direction | Prior art | Composite ball head | Stress decrease |
| 0° | 14.82 | 12 | 19.03% |
| 45° | 14.51 | 12.01 | 17.23% |
| 70° | 17.35 | 15.02 | 13.43% |

It can be seen from Table 1 that under the same load, the maximum stress generated by the composite ball head of the present invention is lower than the maximum stress of the conventional metal ball head hip joint.

The present invention provides the artificial hip joint with the composite ball head, which maintains all advantages of large ball head hip joints, such as increasing the joint mobility, reducing the boundary stress/edging loading, reducing the collision between boundaries of the femoral stem and the acetabular shell, reducing the risk of dislocation of the acetabulum and the ball head, avoiding the negative effect of the small ball head, effectively prolonging the service life of the artificial joint, and enhancing the useful function of the artificial joint. Compared with the conventional surgery, it is easy to implant without increasing any difficulty. The present invention adopts the composite ball head, and the metal ball head is wrapped with the wear-resistant non-metal shell, wherein the support area is large, the contact stress is reduced, the bearing capacity is strong, stable and reliable. It is not easy to damage, and the service life of the artificial joint is prolonged.

The present invention can be used in conjunction with any conventional femoral stem, which is easy to implement, and has a wide range of versatility. The present invention can also completely eliminate the contact surface between the metal ball head and the metal femoral stem, and does not introduce any metal-to-metal interface, thereby completely eliminating the origin of metal debris and metal ions.

The above describes only preferred embodiments of the present invention, and it should be noted that those skilled in the art can make various modifications and improvements without departing from the inventive concept, such are within the scope of the present invention.

1: An artificial hip joint with a composite ball head, comprising an artificial acetabulum and the composite ball head; wherein an internal surface of the artificial acetabulum cooperates with an external surface of the composite ball head, and the composite ball head is rotatable within the artificial acetabulum; the internal surface of the artificial acetabulum is directly in contact with the composite ball head without a liner therebetween; the composite ball head comprises a metal ball head and a non-metallic shell wrapped around an external surface of the metal ball head, the external surface of the composite ball head completely matches with the internal surface of the non-metallic shell;
wherein the metal ball head comprises a spherical end portion and a cylindrical extension portion; a cavity formed by the internal surface of the non-metallic shell matches with an external shape of the metal ball head;
wherein the metal ball head has a concave tapered space which cooperates with a top end of a femoral stem, and the metal ball head is combined with the femoral stem through the concave tapered space, so as to be installed at a thigh bone;
or the metal ball head is integrally provided with a femoral stem.

2: The artificial hip joint, as recited in claim 1, wherein the artificial acetabulum is made of a metal material.

3: The artificial hip joint, as recited in claim 1, wherein the non-metallic shell of the composite ball head is made of a wear resistant non-metallic material.

4: The artificial hip joint, as recited in claim 1, wherein the non-metallic shell of the composite ball head is made of a high molecular weight polyethylene material or a PEEK (polyether ether ketone) material.

5: The artificial hip joint, as recited in claim 1, wherein the internal surface of the artificial acetabulum is a smooth surface.

6: The artificial hip joint, as recited in claim 1, wherein an external surface of the artificial acetabulum is coated with a coating which facilitates bone ingrowth.

7: The artificial hip joint, as recited in claim 1, wherein a size of the composite ball head is close to, identical to, or exceeds a size of a patient femoral head according to a size of the artificial acetabulum.

8: The artificial hip joint, as recited in claim 1, wherein the metal ball head comprises a protruding member, and the protruding member is in contact with an external bottom end of the non-metallic shell.

9: The artificial hip joint, as recited in claim 2, wherein a size of the composite ball head is close to, identical to, or exceeds a size of a patient femoral head according to a size of the artificial acetabulum.

10: The artificial hip joint, as recited in claim 3, wherein a size of the composite ball head is close to, identical to, or exceeds a size of a patient femoral head according to a size of the artificial acetabulum.

11: The artificial hip joint, as recited in claim 4, wherein a size of the composite ball head is close to, identical to, or exceeds a size of a patient femoral head according to a size of the artificial acetabulum.

12: The artificial hip joint, as recited in claim 5, wherein a size of the composite ball head is close to, identical to, or exceeds a size of a patient femoral head according to a size of the artificial acetabulum.

13: The artificial hip joint, as recited in claim 6, wherein a size of the composite ball head is close to, identical to, or exceeds a size of a patient femoral head according to a size of the artificial acetabulum.

14: The artificial hip joint, as recited in claim 2, wherein the metal ball head comprises a protruding member, and the protruding member is in contact with an external bottom end of the non-metallic shell.

15: The artificial hip joint, as recited in claim 3, wherein the metal ball head comprises a protruding member, and the protruding member is in contact with an external bottom end of the non-metallic shell.

16: The artificial hip joint, as recited in claim 4, wherein the metal ball head comprises a protruding member, and the protruding member is in contact with an external bottom end of the non-metallic shell.

17: The artificial hip joint, as recited in claim 5, wherein the metal ball head comprises a protruding member, and the protruding member is in contact with an external bottom end of the non-metallic shell.

18: The artificial hip joint, as recited in claim 6, wherein the metal ball head comprises a protruding member, and the protruding member is in contact with an external bottom end of the non-metallic shell.
